(19) United States
(12) Patent Application Publication
(10) Pub. No.: US 2022/0226037 A1
(43) Pub. Date: Jul. 21, 2022

(54) Electrosurgical Electrode and Electrosurgical Tool for Conveying Electrical Energy

(71) Applicant: Stryker European Operations Limited, Cork (IE)

(72) Inventors: Kevin Buckley, Cork (IE); Gerard Nunan, Cork (IE); Kevin Manley, Cork (IE); Scott McFarland, Greenisland (IE); Micheal Burke, Cork (IE)

(21) Appl. No.: 17/615,461

(22) PCT Filed: May 29, 2020
(86) PCT No.: PCT/IB2020/000441

§ 371 Date: Nov. 30, 2021

Related U.S. Application Data
(60) Provisional application No. 62/854,803, filed on May 30, 2019.
(60) Provisional application No. 62/934,489, filed on Nov. 12, 2019.

(51) Int. Cl. A61B 18/14

(52) U.S. Cl. CPC A61B 18/14; A61B 2018/00083

(57) ABSTRACT

An electrosurgical tool for conveying electrical energy comprising an elongated electrode extending in an axial direction from a proximal electrode end to a distal electrode end. The distal electrode end defining a working end configured for cutting or coagulation of tissue by way of electrical energy received by the electrosurgical tool. At least one layer of an insulation material covering an outer surface of the working end so that a portion of the outer surface of the working end is not covered by the insulation material. When electrical energy is provided to the elongated electrode, current is only conducted through an exposed portion of the outer surface of the working end. At least one layer of the insulation material prevents current from straying from the outer surface of the working end covered with the insulation material.

### CROSS-REFERENCE TO RELATED APPLICATIONS

The present application claims the benefit of priority of U.S. Provisional Application No. 62,934,489 filed on Nov. 12, 2019 and U.S. Provisional Application No. 62/854,803 filed on May 30, 2019, the contents of which are hereby incorporated by reference in their entirety.

### FIELD

The present disclosure generally relates to methods and apparatus for conveying electrical energy and, more particularly, to an electrosurgical tool having an elongated electrode that may be used for cutting tissue or coagulating tissue using electrical energy that is received by the elongated electrode.

### BACKGROUND

Electrosurgery involves applying a radio frequency (RF) electric current (also referred to as electrical energy) to biological tissue to cut, coagulate, or modify the biological tissue during an electrosurgical procedure. Specifically, an electrosurgical generator generates and provides the electric current to an active electrode, which applies the electric current (and, thus, electrical power) to the tissue. The electric current passes through the tissue and returns to the generator via a return electrode (also referred to as a “dispersive electrode”) in monopolar system or a second active electrode in a bipolar system. As the electric current passes through the tissue, an impedance of the tissue converts a portion of the electric current into thermal energy (e.g., via the principles of resistive heating), which increases a temperature of the tissue and induces modifications to the tissue (e.g., cutting, coagulating, ablating, and/or sealing the tissue).

For example, when tissue temperatures reach approximately 55 degrees Celsius (C), cells in the vicinity die. If more current is applied, the temperature keeps rising, the dead cells become desiccated and the proteins coagulate. If yet more current is applied and heat rises still further (above 100° C.), the remnants of the tissue will be vaporized.

### SUMMARY

In an example, an electrosurgical electrode for conveying electrical energy is described. The electrosurgical electrode includes a proximal electrode end configured to receive electrical energy from an electrosurgical tool, a distal electrode end, and a working end portion between the proximal electrode end and the distal electrode end. The working end portion is configured for cutting or coagulation of tissue using the electrical energy that is received by the proximal electrode end. The electrosurgical electrode further includes a first lateral surface, a second lateral surface opposite the first lateral surface, a first face extending between the first lateral surface and the second lateral surface on a first side of the electrosurgical electrode, and a second face extending between the first lateral surface and the second lateral surface on a second side of the electrosurgical electrode that is opposite the first side.

Additionally, the electrosurgical electrode incudes one or more apertures extending entirely through a thickness of the elongated electrode between the first face and the second face. The electrosurgical electrode also includes at least one layer of an insulation material is coupled to an outer surface of the working end so that a first portion of the outer surface is covered by the at least one layer of insulation material and a second portion of the outer surface is not covered by the at least one layer of insulation material. The at least one layer of insulation material is configured to prevent applying electric current from the first portion of the outer surface to a tissue of a patient. The at least one layer of insulation material is coupled to the outer surface at the one or more apertures.

In another example, an electrosurgical electrode for conveying electrical energy is described. The electrosurgical electrode includes a proximal electrode end configured to receive electrical energy from an electrosurgical tool, a distal electrode end, and a working end portion between the proximal electrode end and the distal electrode end. The working end portion is configured for cutting or coagulation of tissue using the electrical energy that is received by the proximal electrode end. The electrosurgical electrode further includes a first lateral surface, a second lateral surface opposite the first lateral surface, a first face extending between the first lateral surface and the second lateral surface on a first side of the electrosurgical electrode, and a second face extending between the first lateral surface and the second lateral surface on a second side of the electrosurgical electrode that is opposite the first side. The electrosurgical electrode also includes a plurality of teeth on at least one of the first lateral surface or the second lateral surface, wherein the plurality of teeth can each taper to a respective tip point.

The features, functions, and advantages that have been discussed can be achieved independently in various examples or may be combined in yet other examples further details of which can be seen with reference to the following description and drawings.

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying drawings, wherein:

FIG. 1 illustrates an electrosurgical system for performing electrosurgery, according to an example implementation.

FIG. 2 illustrates an electrosurgical pencil for use in an electrosurgical system, such as the system illustrated in FIG. 1.

FIG. 3 illustrates a side view of an elongated electrosurgical electrode, according to an example implementation.

FIG. 4A illustrates a cross-sectional view of the elongated electrosurgical electrode illustrated in FIG. 3.

FIG. 4B illustrates another cross-sectional view of the elongated electrosurgical electrode illustrated in FIG. 3.

FIG. 5 illustrates a side view of an elongated electrosurgical electrode, according to an example implementation.

FIG. 6 illustrates a perspective view of an elongated electrosurgical electrode, according to an example implementation.

FIG. 7 illustrates another perspective view of the elongated electrosurgical electrode illustrated in FIG. 6.

FIG. 8 illustrates a perspective view of an elongated electrosurgical electrode, according to an example implementation with seamless insulating layer applied.

FIG. 9 illustrates another perspective view of the elongated electrosurgical electrode illustrated in FIG. 8 with seamless insulating material applied.

FIG. 10 illustrates a perspective view of an elongated electrosurgical electrode, according to an example implementation.

FIG. 11 illustrates another perspective view of the elongated electrosurgical electrode illustrated in FIG. 10.

FIG. 12 illustrates a perspective view of an elongated electrosurgical electrode, according to an example implementation.

FIG. 13 illustrates another perspective view of the elongated electrosurgical electrode illustrated in FIG. 12.

FIG. 14 illustrates another electrosurgical system for performing electrosurgery, according to an example implementation.

FIG. 15A illustrates a perspective view of the electrosurgical electrode, according to an example implementation.

FIG. 15B illustrates a plan view of the electrosurgical electrode illustrated in FIG. 15A.

FIG. 15C illustrates a first side view of the electrosurgical electrode illustrated in FIG. 15A.

FIG. 15D illustrates a second side view of the electrosurgical electrode illustrated in FIG. 15A.

FIG. 16A illustrates a perspective view of an electrosurgical electrode, according to an example implementation.

FIG. 16B illustrates a plan view of the electrosurgical electrode illustrated in FIG. 16A.

FIG. 16C illustrates a side view of the electrosurgical electrode 1600 illustrated in FIG. 16A.

FIG. 17A illustrates a plan view of an electrosurgical electrode, according to another example.

FIG. 17B illustrates a cross-sectional view of the electrosurgical electrode shown in FIG. 17A, according to an example.

### DETAILED DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

While performing electrosurgery, an electrosurgical electrode may apply to tissue some stray electrical current, which is not used for a desired cutting or coagulation of the tissue. It would be beneficial to perform electrosurgery with reduced stray current. It would also be beneficial to reduce stray current while having a desired current flow only through a desired cutting zone so that there will also be less smoke created, thereby further reducing undesired airborne artifacts. The disclosed electrosurgical electrodes may be utilized to focus and direct electrical current to a desired tissue target while also help to reduce stray or undesired non-cutting current.

Within examples, the electrosurgical electrodes of the present disclosure can focus and direct the electrical current in this manner due to one or more geometrical features of the electrosurgical electrode and/or one or more layers of an insulation material covering select portions of the electrosurgical electrodes. For instance, the electrosurgical electrodes can include geometrical features at one or more edges to assist in increasing a density of the electrical current at the edges. As examples, the geometrical features can include a relatively fine edge (e.g., a relatively sharp edge) and/or a plurality of teeth that each taper to a relatively fine tip. Example cutting edges may be machined or designed along at least a portion of the blade so as to exhibit certain desired cleaving or cutting edges that concentrate electrical current towards a desired tissue target.

As used herein, the term “insulation material” means a material that is suitable to cover the portion of an outer surface of the electrosurgical electrode and prevent the application of electrical energy from the portion of the outer surface to a tissue of a patient. Accordingly, by applying the insulation material to a first portion of the electrosurgical electrode and omitting the insulation material from a second portion of the electrosurgical electrode, the electrical current that is applied to the tissue of the patient can be focused at the second portion of the electrosurgical electrode. In an implementation, the second portion of the electrosurgical electrode can be at least one edge of the electrosurgical electrode.

With the geometrical features and/or the selectively applied insulation material, the electrosurgical electrodes disclosed herein can reduce stray current that is current not used for the desired cutting or coagulation of the targeted tissue. The electrosurgical electrodes can cause less collateral damage to tissue surrounding the targeted tissue zone. As another advantage of reducing stray current and having the desired current flow through only the desired cutting zone is that there will also be less smoke created, thereby further reducing undesired airborne contaminants.

The electrosurgical electrodes disclosed herein can also provide enhanced cutting efficiencies. Cutting efficiencies may be enhanced with an electrosurgical electrode blade that facilitates a desired placement of the insulating material along an outer surface of the blade by way of one or more apertures. One or more apertures, openings, slots and/or holes provided by the electrosurgical electrode blade will be used to help secure the insulation material along the outer surface of the blade. One intention of such apertures etc. is to allow insulating material on one face to join with insulating material on the other face and create a seamless ring of insulation that will not lift or delaminate.

One or more apertures may extend along a portion of the length of the blade. One or more apertures etc. may be provided near an edge of the blade. Alternatively or in addition, one or more apertures etc. may be provided at alternative locations, away from an edge of the blade. As one example, an aperture may comprise a slot having a thickness of approximately 125 microns.

As described above, the electrosurgical electrode can include at least one layer of insulation material that covers a select portion of the outer surface of the electrosurgical electrode. Covering the select portion of the outer surface with the at least one layer of insulation material presents a technical challenge in that the insulation material may decouple from the electrosurgical electrode during or after an electrosurgical procedure. For example, in some instances, when the at least one layer of insulation material does not extend around an entire circumference of the electrosurgical electrode, the at least one layer of insulation material can have a free edge that can contact the tissue during the electrosurgical procedure. When the tissue contacts the free edge of the at least one insulation layer, the tissue can apply a force to the free edge that causes the free edge to decouple from the outer surface of the electrosurgical electrode.

Within examples, the electrosurgical electrodes described herein can address this technical problem associated with covering the select portion of the electrosurgical electrode with the at least one layer of insulation material. Specifically, within examples, the electrosurgical electrodes can include one or more apertures that extend entirely through a thickness of the electrosurgical electrode such that the at least one layer of insulation material can be received and/or extend through the one or more apertures. In this way, the one or more apertures can provide a passage through which the at least one layer of insulation material can extend so that the at least one layer of insulation material can extend between opposing sides of the electrosurgical electrode (e.g., as a continuous loop of the insulation material).

In this arrangement, when the tissue applies a force to the at least one layer of insulation material, the at least one layer of insulation material is forced against the outer surface of the electrosurgical electrode due to the portion of the at least one layer of insulation material that extends through the one or more apertures. As such, the one or more apertures can help to inhibit or prevent the at least one layer of insulation material from decoupling from the electrosurgical electrode.

Example electrosurgical electrodes described herein can be used with various different types of radio-frequency (RF) electrosurgical systems, including monopolar electrosurgical systems and bipolar electrosurgical systems.

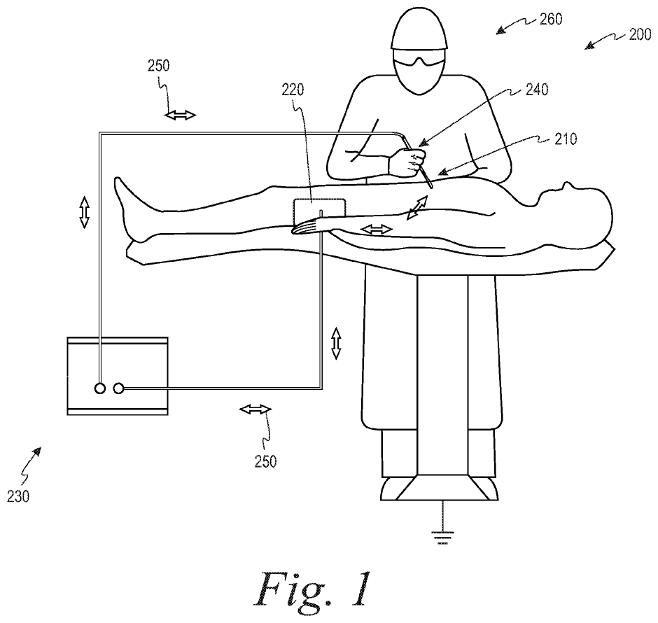

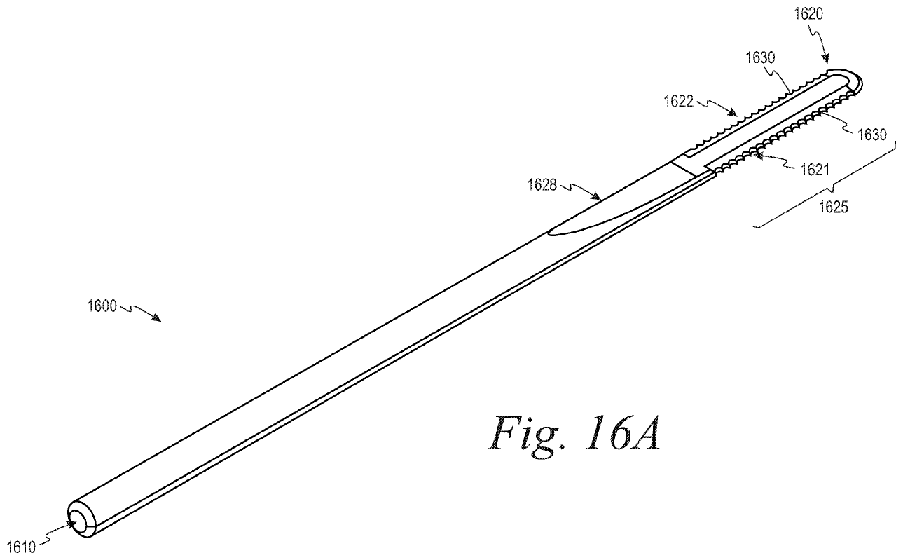

Referring now to FIG. 1, an electrosurgical system 200 is illustrated according to an example. In FIG. 1, the electrosurgical system 200 is a monopolar electrosurgical system. However, as described in further detail below with respect to FIG. 14, the concepts of the present disclosure can be additionally or alternatively implemented in a bipolar electrosurgical system.

As shown in FIG. 1, the electrosurgical system 200 includes an electrosurgical electrode 210, a dispersive electrode 220, a RF generator 230, and an electrosurgical tool 240. The RF generator 230 is configured to generate an electric current 250 that is suitable for performing electrosurgery on a patient. For example, the RF generator 230 can include a power converter circuit that can convert a grid power to electrical energy such as, for example, a RF output power. As an example, the power converter circuit can include one or more electrical components (e.g., one or more transformers) that can control a voltage, a current, and/or a frequency of the electrical energy.

The electrosurgical tool 240 can include the electrosurgical electrode 210, and the electrosurgical tool 240 can include one or more electrical components that are configured to supply the electric current 250 from the RF generator 230 to the electrosurgical electrode 210. As described in further detail below, the electrosurgical electrode 210 can then use the electric current to apply electrical energy to a tissue of the patient.

The dispersive electrode 220 can be coupled to a body of the patient, and the RF generator 230. In this arrangement, the RF generator 230 can supply the electric current to the electrosurgical electrode 210, the electrosurgical electrode 210 can apply the electric current to the tissue, the tissue can conduct the electric current to the dispersive electrode 220, and the dispersive electrode 220 can return the electric current to the RF generator 230.

Within examples, the electrosurgical system 200 can be used for at least one treatment modality selected from a group of modalities including cutting, coagulation, and fulguration. In FIG. 1, a surgeon 260, using the electrosurgical tool 240 (e.g., an electrosurgical pencil) containing the electrosurgical electrode 210, places the electrosurgical electrode 210 adjacent to patient tissue to cut said tissue and coagulate bleeding of a patient.

Current from the electrosurgical electrode 210 develops a high temperature region about an exposed end of the electrosurgical electrode 210 and this affects the tissue. As will be described in detail herein, the disclosed electrosurgical electrode 210 reduces unwanted stray current from the exposed end of the electrosurgical electrode 210 and thereby limits unintended tissue damage/destruction. This also tends to reduce an accumulation of unwanted eschar and smoke (e.g., undesired smoke particles).

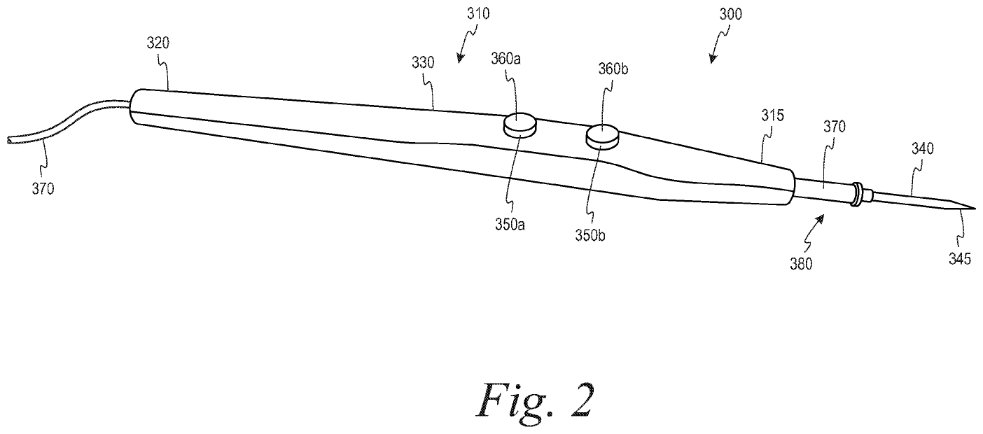

FIG. 2 illustrates close up view of an electrosurgical tool 300 for conveying electrical energy for use in a monopolar electrosurgical system, according to an example. For example, the electrosurgical tool 300 may be used in the monopolar electrosurgical system 200 illustrated in FIG. 1. Alternative electrosurgical tools 300 may be used in bipolar electrosurgical systems, such as the bipolar electrosurgical system 1000 illustrated in FIG. 14 and described herein.

In the illustrated arrangement of FIG. 2, the electrosurgical tool 300 is in the form of an electrosurgical pencil. As such, in FIG. 2, the electrosurgical pencil 310 has an elongated shape that facilitates the user holding the electrosurgical tool 300 in a writing utensil gripping manner. However, the electrosurgical tool 300 can have a different shape and/or a different size in other examples. More generally, the electrosurgical tool 300 can be configured to facilitate a user gripping and manipulating the electrosurgical tool 300 while performing electrosurgery. Therefore, the electrosurgical tool 300 can be manually manipulated by a surgeon to cut or coagulate tissue by way of RF power, as described above.

Referring to FIG. 2, the electrosurgical pencil 310 generally extends from a first or distal end 315 to a second or proximal end 320. The electrosurgical pencil 310 comprises an elongated housing structure 330 that may be used to house certain electrosurgical pencil components. The distal end 315 of the elongated housing structure 330 receives an electrosurgical electrode 340. The electrosurgical electrode 340 may comprise a metal tip 345 that is used to cut, or to coagulate tissue during surgery. In one example, the metal tip 345 comprises a pointed metal tip. In another example, the metal tip 345 may comprise a blade type structure having one or more machined cutting edges as will be described in greater detail herein. An insulating sleeve or an insulating cover 371 may be provided near a proximal portion 380 of the electrosurgical electrode 340. The insulating cover 371 can be made from a material that prevents the electrosurgical electrode 340 from transmitting electrical energy to the tissue via a portion of the electrosurgical electrode 340 that is covered by the insulating cover 371. In one example, the electrosurgical electrode 340 is configured to be removably coupled to the electrosurgical pencil 310.

The elongated housing structure 330 of the electrosurgical tool 300 may also define a plurality of windows or cavities 350a, 350b. These windows or cavities 350a, 350b may be defined to receive one or more human interface devices 360a, 360b. In an example, the elongated housing structure 330 includes a first cavity 350a and a second cavity 350b for receiving a first human interface device 360a and a second human interface device 360b, respectively. As one example, each human interface device 360 a, 360b may be utilized to perform certain electrosurgical functions, such as cutting or coagulating tissue. In one example, the first human interface device 360a can be used to coagulate while the second human interface device 360b can be used to cut. Other human interface device configurations may also be used.

The electrosurgical tool 300 also includes an insulating cable 370 which provides power to the electrosurgical electrode 340. This insulating cable 370 may receive power from an RF generator, such as the RF generators illustrated in FIGS. 1 and 14. Alternatively, the electrosurgical pencil 310 may include an independent power supply such as a self-contained power supply.

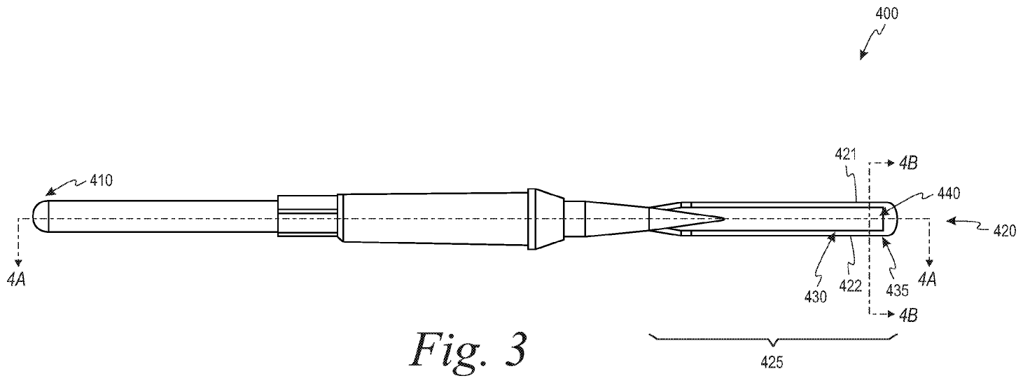

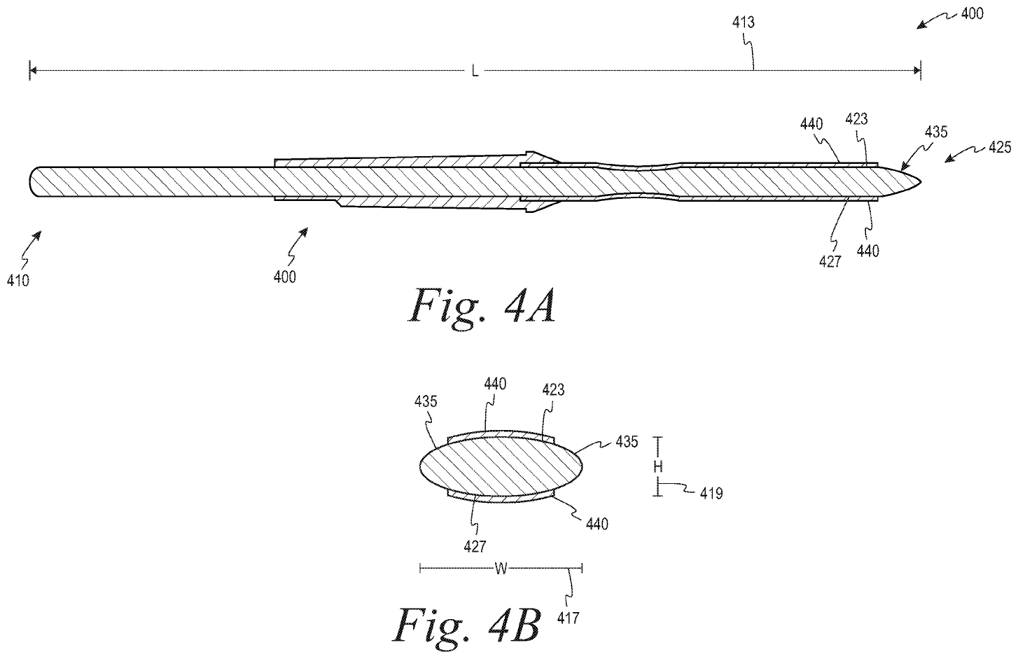

FIG. 3 illustrates an electrosurgical electrode 400 that may be used with the electrosurgical tool 300 illustrated in FIG. 2, according to an example. FIG. 4A illustrates a first cross-sectional view of the electrosurgical electrode 400 illustrated in FIG. 3, and FIG. 4B illustrates a second cross-sectional view of the electrosurgical electrode 400 illustrated in FIG. 3, according to an example.

Referring to FIGS. 3-4B, the electrosurgical electrode 400 extends in an axial direction along a longitudinal axis from a proximal electrode end 410 to a distal electrode end 420. As shown in FIG. 4A, a distance between the proximal electrode end 410 and the distal electrode end 420 can define a length 413 of the electrosurgical electrode 400. In an example, the length 413 between the proximal electrode end 410 and the distal electrode end 420 can be approximately 65 mm to approximately 75 mm. However, alternative distances may also be used.

The electrosurgical electrode 400 also includes a first lateral surface 421 and a second lateral surface 422 extending between the proximal electrode end 410 and the distal electrode end 420. As shown in FIG. 4B, a distance between the first lateral surface 421 and the second lateral surface 422 can define a width 417 of the electrosurgical electrode 400.

The electrosurgical electrode 400 further includes a first major face 423 and a second major face 427 on an opposite side of the electrosurgical electrode 400 relative to the first major face 423. The first major face 423 and the second major face 427 each (i) extend between the proximal electrode end 410 and the distal electrode end 420, and (ii) extend between the first lateral surface 421 and the second lateral surface 422. As shown in FIG. 4B, a distance between the first major face 423 and the second major face 427 can define a thickness 419 of the electrosurgical electrode 400. As shown in FIG. 4B, the thickness 419 can vary over the width 417 of the electrosurgical electrode 400.

In one example, the electrosurgical electrode 400 includes a working end portion 425 between the proximal electrode end 410 and the distal electrode end 420. The working end portion 425 is configured for cutting and/or coagulation of tissue using electrical energy that is received by an electrosurgical tool, such as the electrosurgical tool 300 illustrated in FIG. 2. In addition, the electrosurgical tool may receive such electrical energy by way of a RF power source, such as the RF generator 230 illustrated in FIG. 1 or the RF generator 1100 illustrated in FIG. 14. In one example, the working end portion 425 of the electrosurgical electrode 400 comprises a sharpened or pointed tip at the distal electrode end 420 of the electrosurgical electrode 400. Alternatively, the working end portion 425 may comprise a blade type structure having at least one beveled edge for cutting tissue. Other electrode working end configurations may also be used.

In an example, at least one layer of an insulation material 440 covers a portion of an outer surface 430 of the working end portion 425, and the at least one layer of insulation material 440 does not cover a second portion 435 of the working end portion 425. In this configuration, the second portion 435 of the outer surface 430 of the working end portion 425 remains uncovered by the at least one layer of the insulation material 440. In one example, the working end portion 425 of the electrosurgical electrode 400 may comprise a total surface area of approximately 55 mm² and the insulation material 440 may cover approximately 70 percent to approximately 80 percent of this total surface area (e.g., approximately 42 mm²).

As used herein, the term “insulation material” means a material that is suitable to cover the portion of the outer surface 430 and prevent the application of electrical energy from the portion of the outer surface 430 to a tissue of a patient. In this manner, when electrical energy is provided to the electrosurgical electrode 400, current is substantially conducted to the target tissue only through the exposed select portion 435 of the outer surface 430 of the working end portion 425 of the electrosurgical electrode 400. Similarly, the at least one layer of the insulation material 440 acts to prevent current from straying from the outer surface 430 of the working end portion 425 that is covered with the insulation material 440. As such, the insulation material 440 reduces certain undesired effects that may be caused by stray currents generated by the electrosurgical electrode 400 during electrosurgical procedures. In addition, the build-up of eschar will not affect the performance of an insulated electrode as much as a normal, uninsulated, blade where eschar build-up may occur at a relatively similar thickness over the top of the electrode surface, both insulated and un-insulated. In the case of the former, the electricity is forced through the caked-on eschar because the current will seek a path of least resistance. In the latter, current that is inhibited by eschar will instead flow through another least restrictive current path, and act as stray current flowing through unintended tissue.

In one example, the at least one layer of the insulation material 440 comprises a polymeric material. For example, a thickness of the at least one layer of the insulation material 440 may comprise at least approximately 100 microns of insulation material. In the arrangement shown in FIGS. 3-4B, a single layer of 120 microns of insulation material 440 is provided to substantively cover the working end portion 425 of the electrosurgical electrode 400. However, in alternative electrosurgical electrode arrangements, one or more such layers may be provided along at least one portion of the electrosurgical electrode 400. As just one example, a first portion of the electrosurgical electrode 400 may comprise a first layer of insulation material 440 while a second portion of the electrosurgical electrode 400 may comprise both a first layer and a second layer of insulation material 440.

In one example, the polymeric material comprises a fluorocarbon material. As an example, the fluorocarbon material comprises polytetrafluoroethylene (PTFE). As noted above, the layer of insulation material 440 can have a thickness of at least 100 microns. This range of thicknesses is generally suitable to ensure that the polymeric material(s) prevent the application of electrical current as described above. However, other insulation materials may be additionally or alternatively used. For example, the insulation material 440 can be silicone, poly olefin, and/or polyamide having sufficient thickness to prevent application of electrical energy to the tissue. In general, the thickness of such alternative material(s) is suitable to prevent the application of electrical current and, in some implementations, the thickness may differ from the range of thicknesses described above for polymeric materials.

In some examples, the insulation material 440 can have a constant thickness over an entire surface area of the portion of the outer surface 430 covered by the at least one layer of insulation material 440. The at least one layer of insulation material 440 having a constant thickness can be formed, for instance, by an over-molding process, spray coating, and/or a dip coating the electrosurgical electrode 400 using a mask to prevent the insulation material 440 from coupling to the select portion 435 that is to be exposed. The at least one layer of insulation material 440 having a constant thickness can help to reduce manufacturing complexities and/or help to reduce or prevent dielectric breakdown of the at least one layer of insulation material 440.

In other examples, the insulation material 440 can have a variable thickness such that the thickness of the insulation material changes over the surface area of the portion of the outer surface 430 covered by the at least one layer of insulation material 440. The at least one layer of insulation material 440 having a variable thickness can be formed, for instance, by over-molding, dip coating, spray coating, and/or vapor deposition. In some implementations, the at least one layer of insulation material 440 having a variable thickness can be formed due to variances in a shape of the electrosurgical electrode 400 and as a result of particular manufacturing techniques.

In some examples, the at least one layer of insulation material 440 can include a single layer of a single type of insulation material. In other examples, the at least one layer of insulation material 440 can include a combination of a plurality of insulation materials and/or a plurality of insulation layers. As just one example, a first layer of a first type of insulation material may be provided (e.g., a first layer of a first type of polymeric material) and a second layer of a second type of insulation material may be provided (e.g., a second layer of second type of polymeric material, different than the first type of polymeric material).

In the example shown in FIGS. 3-4B, the second portion 435 that is not covered by the at least one layer of the insulation material 440 is shown with an underlying conductive substrate of the electrosurgical electrode 400 exposed. However, in other examples, the conductive substrate of the electrosurgical electrode 400 can be covered at the second portion 435 by one or more layers of a material (e.g., a non-stick coating) that does not prevent the application of the electrical energy to the tissue. For instance, the second portion 435 can be covered by one or more layers of the materials described above for the insulation material 440, but with a relatively lower thickness that is suitable to allow the electrical energy to pass through the one or more layers of material from the second portion 435 to the tissue.

As described above, the electrosurgical electrode 400 can include at least one layer of insulation material 440 that covers a select portion of the outer surface 430 of the electrosurgical electrode 400. Covering the select portion of the outer surface 430 with the at least one layer of insulation material 440 presents a technical challenge in that the insulation material 440 may decouple from the electrosurgical electrode 400 during or after an electrosurgical procedure. For example, in some instances, when the at least one layer of insulation material 440 does not extend around an entire circumference of the electrosurgical electrode 400, the at least one layer of insulation material 440 can have a free edge that can contact the tissue during the electrosurgical procedure. When the tissue contacts the free edge of the at least one layer of insulation material 440, the tissue can apply a force to the free edge that causes the free edge to decouple from the outer surface 430 of the electrosurgical electrode 400.

Within examples, the electrosurgical electrodes described herein can address this technical problem associated with covering the select portion of the electrosurgical electrode 400 with the at least one layer of insulation material. Specifically, within examples, the electrosurgical electrodes can include one or more apertures that extend entirely through a thickness of the electrosurgical electrode such that the at least one layer of insulation material can be received and/or extend through the one or more apertures. In this way, the one or more apertures can provide a passage through which the at least one layer of insulation material can extend so that the at least one layer of insulation material can extend between opposing sides of the electrosurgical electrode (e.g., as a continuous loop of the insulation material).

In this arrangement, when the tissue applies a force to the at least one layer of insulation material, the at least one layer of insulation material is forced toward the outer surface of the electrosurgical electrode due to the portion of the at least one layer of insulation material that extends through the one or more apertures. As such, the one or more apertures can help to inhibit or prevent the at least one layer of insulation material from decoupling from the electrosurgical electrode.

Additionally, the one or more apertures of the electrosurgical electrode can allow for the at least one layer of insulation material to be formed on the outer surface using manufacturing techniques that may be unsuitable for prior coatings on the electrosurgical electrode (e.g., a non-stick coating). For instance, the one or more apertures can allow for the insulation material to be a solid structure that is coupled around a portion of the electrosurgical blade in a manner that allows for some play between the insulation material and an outer surface of the electrosurgical electrode.

The one or more apertures of the electrosurgical electrode can additionally or alternatively simplify manufacturing and/or reduce a cost to manufacture the electrosurgical electrode. For instance, some existing electrosurgical electrodes that include a coasting (e.g., a non-stick coating) may be manufactured by a process that involves texturing a substantial portion of the outer surface of the electrosurgical electrode before coating the electrosurgical electrode. In some implementations, the surface texturing process is performed to help adhere the coating to the outer surface of the electrosurgical electrode. The surface texturing process can include, for instance, an acid etching and/or a sand blasting process to form and/or enhance microscale and/or nanoscale peaks and valleys on the outer surface of the electrosurgical electrode. Because the one or more apertures can assist in coupling the insulation material to the electrosurgical electrode, a process for manufacturing the electrosurgical electrode can optionally omit the surface texturing process.

However, in some examples, a manufacturing process for forming the electrosurgical electrodes described herein can include the above-described surface texturing process to further enhance engagement between the outer surface of the electrosurgical electrode and the insulation material. Additionally or alternatively, the process for manufacturing the electrosurgical electrode can include forming a textured surface on an inner surface within the one or more apertures. This can, for example, help to improve the engagement between the insulation material and the outer surface of the electrosurgical electrode in the one or more apertures. The one or more apertures described herein can be incorporated in any and all of the examples illustrated in the drawings and described herein. In some examples described above and below, the one or more apertures and/or the insulation material may not be explicitly illustrated in the drawings to help more clearly show and describe other features. However, the one or more apertures and/or the at least one layer of insulation material described and/or illustrated for any example herein can be incorporated in any other example described and illustrated in the present disclosure.

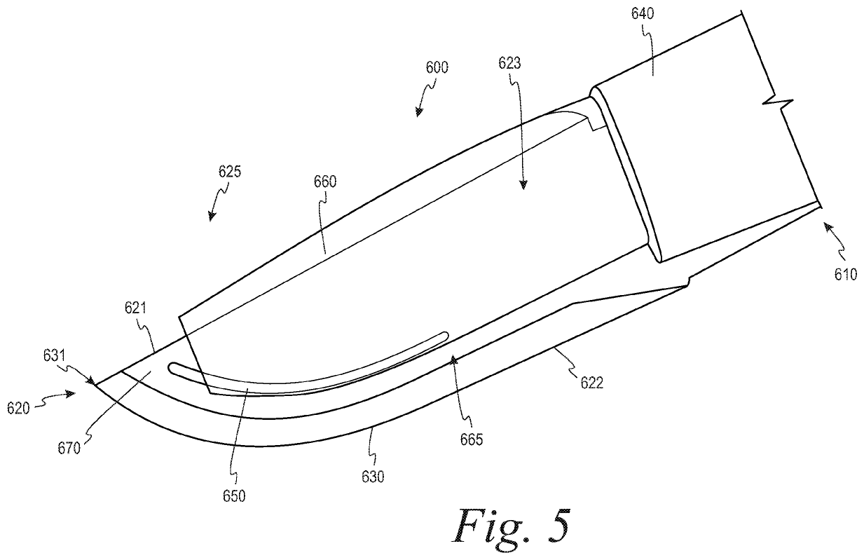

FIG. 5 illustrates an electrosurgical electrode 600 for use with an electrosurgical tool for conveying electrical energy, such as the electrosurgical tool 300 illustrated in FIG. 2, according to an example. As will be described, this electrosurgical electrode 600 may be used for both cutting and coagulation.

Similar to the electrosurgical electrode 400 described above, the electrosurgical electrode 600 extends in an axial direction along a longitudinal axis from a proximal electrode end 610 to a distal electrode end 620. The electrosurgical electrode 600 also includes a first lateral surface 621 and a second lateral surface 622 extending from the proximal electrode end 610 to the distal electrode end 620. The electrosurgical electrode 600 further includes a first major face 623 and a second major face (not shown in FIG. 5) that each (i) extend between the proximal electrode end 610 and the distal electrode end 620, and (ii) extend between the first lateral surface 621 and the second lateral surface 622. In this arrangement, the electrosurgical electrode 600 has a length, a width, and a thickness that are defined as described above.

In FIG. 5, the first lateral surface 621 of the electrosurgical electrode 600 comprises a smooth or generally linear surface. The second lateral surface 622 of the electrosurgical electrode 600 defines a sharp or a machined beveled surface that defines a cutting edge 630. In one arrangement, the cutting edge 630 will not be sharp enough to mechanically cut tissue but will have a fine edge that will concentrate the electricity. As just one example, the fine edge may have an edge thickness in the range of approximately 70 microns to approximately 200 microns. A curved surface along with the first lateral surface 621 can further define a finer tip 631 of the electrosurgical electrode 600.

The second lateral surface 622 includes the cutting edge 630. The cutting edge 630 may be configured for cutting and for coagulation of tissue by way of electrical energy that is received by the conductive electrode 600 as explained herein with respect to the electrosurgical systems illustrated in FIGS. 1 and 14. Near the proximal electrode end 610, an insulating member 640 is provided in the form of a sleeve or cover. For example, such an insulating member 640 may comprise an insulating heat-shrink wrapping. The insulating member 640 can be formed from an insulation material that prevents the transfer of electrical energy to a tissue at the portion of the electrosurgical electrode 600 that is covered by the insulating member 640

In this example, the electrosurgical electrode 600 further defines an aperture 650. In the example shown in FIG. 5, the aperture 650 is formed as a slot that passes through a thickness of the electrosurgical electrode 600. As one example, the thickness of the electrosurgical electrode 600 may range from approximately 0.45 mm and approximately 0.25 mm. However, alternative thicknesses may also be used. In this illustrated arrangement, the aperture 650 propagates along the length and also along the curvature defined by the bottom or cutting edge 630. In the electrosurgical electrode 600 shown in FIG. 5, the first aperture 650 has a generally constant thickness for receiving an insulation material 660. However, in alternative arrangements, the aperture 650 may comprise a non-constant thickness.

This aperture 650 is configured to receive an insulation material 660, such as the insulation material illustrated and described herein with respect to FIGS. 3-4. In this example, the insulation material 660 may be installed or wrapped along an outer surface 670 of the electrosurgical electrode 600 so that only the cutting edge 630 of an outer surface potion of a working end portion 625 remains uncovered by the insulation material 660. For example, a portion 665 of the outer surface 670 of the cutting edge 630 of the electrosurgical electrode 600 remains uncovered by the insulation material 660.

Although the electrosurgical electrode 600 includes only the single aperture 650 illustrated in FIG. 5, the electrosurgical electrode 600 may be utilized with alternative configurations. As just one example, the electrosurgical electrode 600 may define more than one aperture 650. In an example conductive electrode comprising two or more apertures 650, the apertures 650 can have similar geometrical configurations or different geometrical configurations. For example, a conductive electrode comprising a plurality of apertures 650 may comprise apertures 650 having a substantially same thickness but may have varying lengths. Similarly, the aperture 650 can include a plurality of slots that have a substantially similar length but may have varying thicknesses. Alternative geometrical aperture 650 configurations may also be used, such as circular, triangular, oval, trapezoidal, or semi-circular slot configurations.

In the example shown in FIG. 5, the cutting edge 630 comprises a beveled edge and may extend along the entire length of the conductive electrode blade portion. In this example, the length of the conductive blade portion extends first horizontally and then curves towards a distal most tip portion 631 of the blade, thus providing an enhanced cutting edge. Alternative cutting edge configurations may also be utilized, such as a paddle-shaped electrode comprising at least one cutting edge.

As illustrated in FIG. 5, the electrosurgical electrode 600 comprises at least one layer of insulation material 660 provided along an outer surface of the working end portion 625 so that only a select portion 665 of the outer surface 670 of the working end portion 625 is exposed. As such, when electrical energy is provided to the electrosurgical electrode 600, current is only allowed to be conducted through the exposed portion 665 of the outer surface 670 of the distal electrode end 620. Consequently, the at least one layer of insulation material 660 inhibits or prevents stray current from flowing through the outer surface 670 of the working end portion 625 that is covered with the insulation material 660.

In FIG. 5, the portion 665 of the outer surface 670 of the electrosurgical electrode 600 that is exposed includes the cutting edge 630 and at least a portion of the outer surface 670 on the first major face 623 and the second major face. As shown in FIG. 5, the portion 665 of the outer surface 670 of the electrosurgical electrode 600 that is exposed can additionally or alternatively include the tip 631 of the electrosurgical electrode 600.

In one example, the insulation material 660 illustrated in FIG. 5 comprises a polymeric material. This polymeric material may comprise a fluorocarbon material. In one example, the fluorocarbon material comprises polytetrafluoroethylene (PTFE). Alternative insulation/polymeric materials may also be used. In one example, a thickness of the insulation material 660 comprises at least approximately 100 microns. In one example, the cutting edge 630 of the working end portion 625 comprises a longitudinal cutting edge. The longitudinal cutting edge of the working end portion 625 may extend along an entire length of the working end portion 625.

In some examples, the at least one layer of insulation material 660 can a coating. In other examples, the at least one layer of insulation material 660 can be a solid structure that is coupled around a portion of the electrosurgical electrode 600 in a manner that allows for some play between the at least one layer of insulation material 660 and the outer surface 670 of the electrosurgical electrode 600. For instance, the at least one layer of insulation material 660 can form a continuous loop that extends through the aperture 650.

In some implementations, the at least one layer of insulation material 660 can be coupled to the outer surface 670 only by the engagement between the at least one layer of insulation material 660 and the outer surface 670 at the aperture 650. This can be in contrast to alternative implementations in which the at least one layer of insulation material is adhered and/or bonded to the outer surface 670 at the first face 616 and/or the second face.

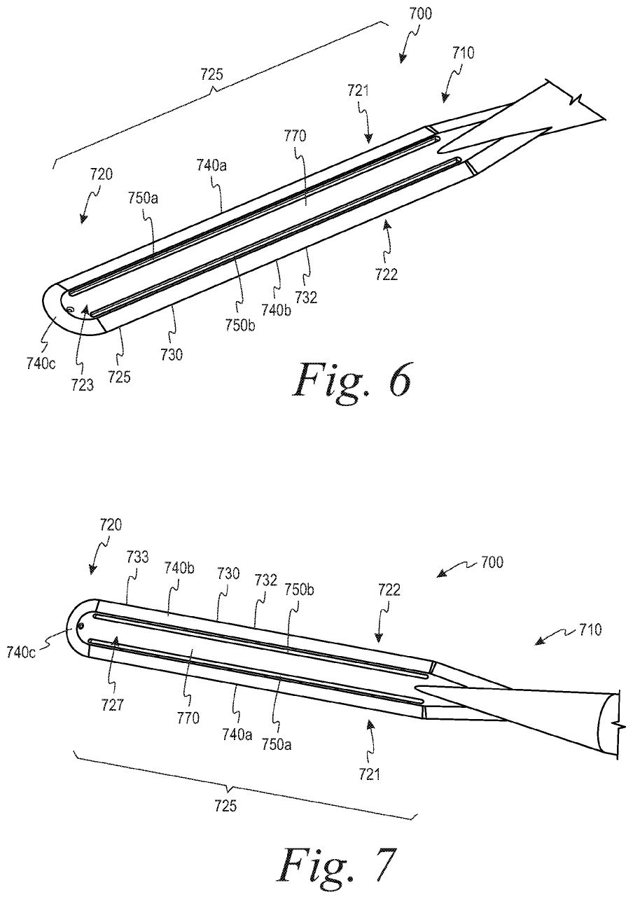

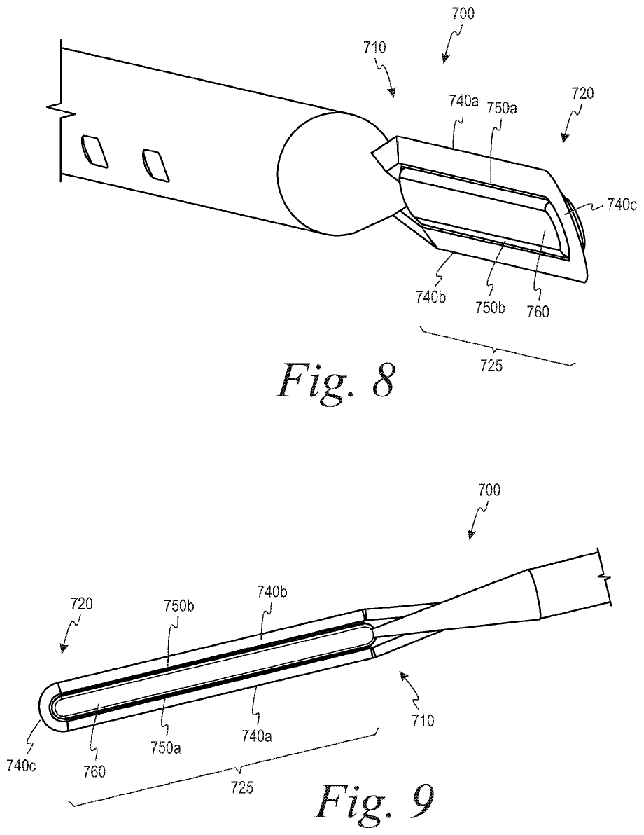

FIG. 6 illustrates a perspective view of an elongated electrosurgical electrode 700, according to another example. The elongated electrosurgical electrode 700 may be used with an electrosurgical tool for conveying electrical energy, such as the electrosurgical tool 300 illustrated in FIG. 2. FIG. 7 illustrates another perspective view of the elongated electrosurgical electrode 700 illustrated in FIG. 6.

Similar to the electrosurgical electrodes 400, 500, 600 described above, the electrosurgical electrode 700 extends in an axial direction along a longitudinal axis from a proximal electrode end 710 to a distal electrode end 720. The electrosurgical electrode 700 also includes a first lateral surface 721 and a second lateral surface 722 extending from the proximal electrode end 710 to the distal electrode end 720. The electrosurgical electrode 700 further includes a first major face 723 and a second major face 727 that each (i) extend between the proximal electrode end 710 and the distal electrode end 720, and (ii) extend between the first lateral surface 721 and the second lateral surface 722. In this arrangement, the electrosurgical electrode 700 has a length, a width, and a thickness that are defined as described above.

The first major face 723 of the electrosurgical electrode 700 (FIG. 6) includes a smooth or generally linear surface. The second major face 727 of the electrosurgical electrode 700 (FIG. 7) also comprises a smooth or general linear surface. In an arrangement, the first major face 723 and the second major face 727 are configured parallel to one another and are tapered toward one another and meet so as to define a sharp or a machined beveled outer electrode perimeter 733. This outer electrode perimeter 733 defines a cutting edge 730 that extends along the perimeter 733 of the electrosurgical electrode 700. In one arrangement, the cutting edge 730 will not be sharp enough to mechanically cut tissue but will comprise a fine edge 732 that will concentrate the electricity. As just one example, the fine edge 732 may have an edge thickness in the range of approximately 70 to approximately 200 microns. The fine edge 732 may be configured for cutting and for coagulation of tissue by way of electrical energy that is received by the conductive electrode 700 as explained herein with respect to the electrosurgical systems illustrated in FIGS. 1 and 14.

In this example, the electrosurgical electrode 700 further defines a first aperture 750a and a second aperture 750b. The first aperture 750a comprises a first slot that passes through the thickness of the electrosurgical electrode 700. As just one example, the thickness of the electrosurgical electrode 700 may range from approximately 0.45 mm and approximately 0.25 mm. However, alternative thicknesses may also be used. In this illustrated arrangement, the first aperture 750a extends along a length defined by a first portion 740a of the cutting edge 730. In the electrosurgical electrode 700 shown in FIGS. 6-7, the first aperture 750a has a generally constant thickness for receiving an insulation material as described herein. However, in alternative arrangements, the first aperture 750a may comprise a non-constant thickness.

Similarly, in this illustrated example, the second aperture 750b comprises a second slot that passes through the thickness of the electrosurgical electrode 700. In this illustrated arrangement, the second aperture 750b propagates along a length defined by a second portion 740b of the cutting edge 730. In the electrosurgical electrode 700 shown in FIGS. 6-7, the second aperture 750b has a generally constant thickness for receiving an insulation material as described herein. However, in alternative arrangements, the second aperture 750b may comprise a non-constant thickness.

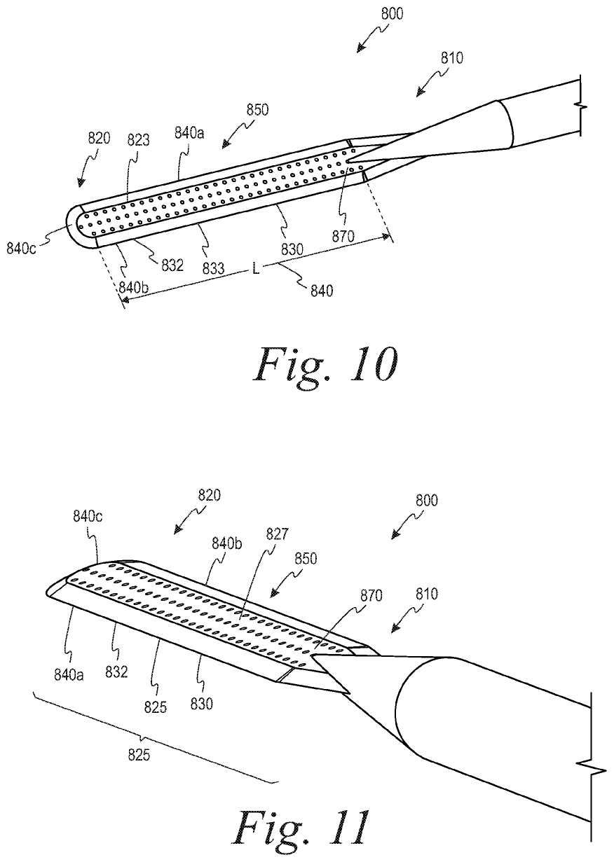

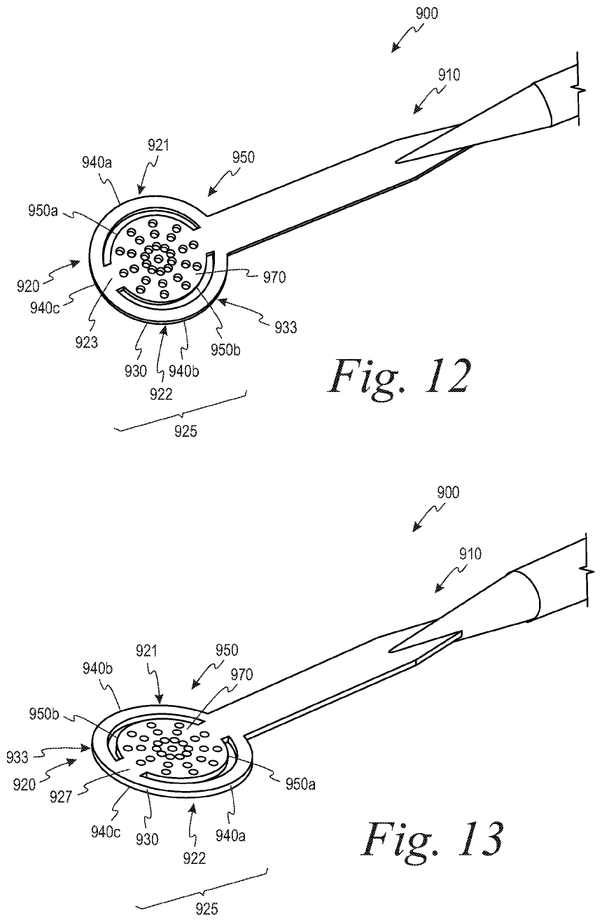

The first aperture 750a and the second aperture 750b are configured to receive an insulation material, such as the insulation material illustrated and described herein with respect to FIGS. 3-5. For example, FIG. 8 illustrates a perspective view of the electrosurgical electrode 700 comprising an insulation material 760. FIG. 9 illustrates another perspective view of the electrosurgical electrode 700 illustrated in FIG. 8. In this illustrated example, the insulation material 760 may be coupled to or wrapped along an outer surface 770 of the electrosurgical electrode 700 (See, FIGS. 6 and 7) so that only the first portion 740a of the cutting edge 730, the second portion 740b of the cutting edge 730, and a third portion 740c of the cutting edge 730 of the outer portion of a working end portion 725 remains uncovered by the insulation material 760.

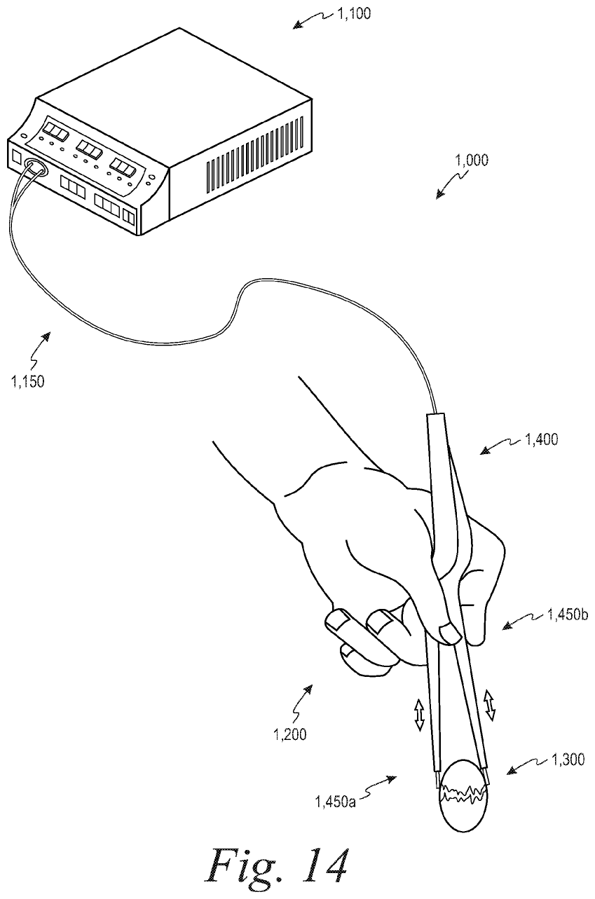

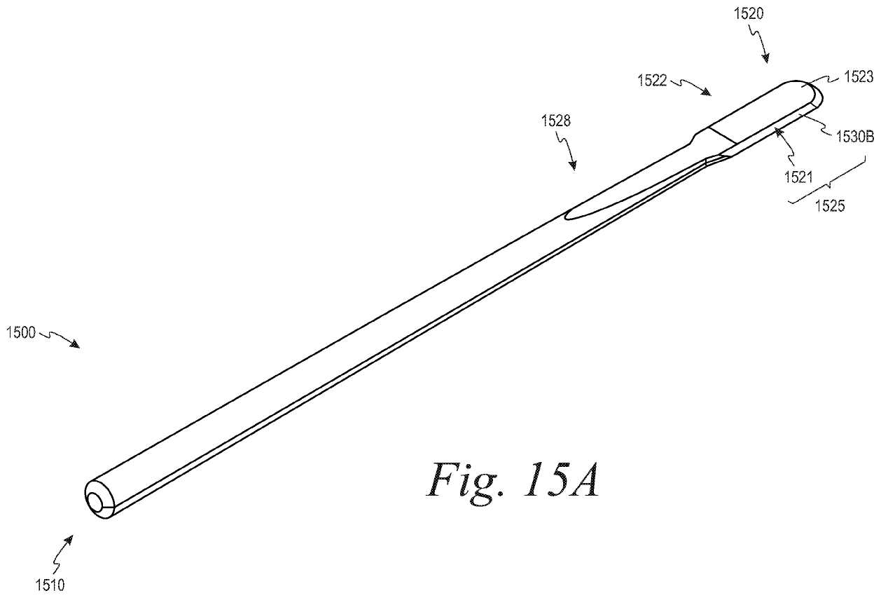

FIG. 10 illustrates a perspective view of an elongated electrosurgical electrode 800, according to an example implementation. FIG. 11 illustrates another perspective view of the elongated electrosurgical electrode 800 illustrated in FIG. 10.

Similar to the electrosurgical electrodes 400, 500, 600, 700 described above, the electrosurgical electrode 800 extends in an axial direction along a longitudinal axis from a proximal electrode end 810 to a distal electrode end 820. The electrosurgical electrode 800 also includes a first lateral surface 821 and a second lateral surface 822 extending from the proximal electrode end 810 to the distal electrode end 820. The electrosurgical electrode 800 further includes a first major face 823 and a second major face 827 that each (i) extend between the proximal electrode end 810 and the distal electrode end 820, and (ii) extend between the first lateral surface 821 and the second lateral surface 822. In this arrangement, the electrosurgical electrode 800 has a length, a width, and a thickness are defined as described above.

The first major face 823 of the electrosurgical electrode 800 (FIG. 10) comprises a smooth or generally linear surface. The second major face 827 of the electrosurgical electrode 800 (FIG. 11) also comprises a smooth or general linear surface. In an arrangement, the first major face 823 and the second major face 827 are configured parallel to one another and are tapered toward one another and meet so as to define a sharp or a machined beveled outer electrode perimeter 833. This outer electrode perimeter 833 defines a cutting edge 830 that extends along the perimeter 833 of the electrosurgical electrode 800. In one arrangement, the cutting edge 830 will not be sharp enough to mechanically cut tissue but will comprise a fine edge 832 that will concentrate the electricity. As just one example, the fine edge 832 may have an edge thickness in the range of approximately 70 to approximately 200 microns. Preferably, this fine edge 832 may be configured for cutting and for coagulation of tissue by way of electrical energy that is received by the conductive electrode 800 as explained herein with respect to the electrosurgical systems illustrated in FIGS. 1 and 14.

In this example, the electrosurgical electrode 800 further defines a plurality of apertures 850 that pass through a thickness of the electrosurgical electrode 800. As just one example, the thickness of the electrosurgical electrode 800 may range from approximately 0.45 mm and approximately 0.25 mm. However, alternative thicknesses may also be used. In this illustrated arrangement, the plurality of apertures 850 are configured in an ordered series or ordered arrangement (e.g., an array of circular apertures arranged in a plurality of rows) that propagates along a length L 840 of the cutting edge 830. However, alternate aperture arrangements could also be used, such as a plurality of apertures configured in a non-ordered series or non-ordered arrangement that propagates along the length L 840 or at least a portion of the length L 840 of the cutting edge 830 (See, FIG. 10).

In the electrosurgical electrode 800 shown in FIG. 10, each of the plurality of apertures 850 comprises a circular aperture and each circular aperture comprises a uniform or constant circumference or radius. However, in alternative circular aperture arrangements, one or more of the circular apertures may comprise a non-uniform or non-constant circumference or radius.

The plurality of apertures 850 are configured to receive an insulation material, such as the insulation material illustrated and described herein with respect to FIGS. 3-4 and as described generally with respect to FIGS. 8 and 9. In such an example, the insulation material may be installed or wrapped along an outer surface 870 of the electrosurgical electrode 800 so that only the first portion 840a of the cutting edge 830, the second portion 840b of the cutting edge 830, and a third portion 840c of the cutting edge 830a of the outer portion of a working end portion 825 remains uncovered by the insulation material, similar to the elongated electrode configuration illustrated in FIGS. 8 and 9. One or more apertures provided by the electrosurgical electrode 800 will be used to help secure the insulation material along the outer surface 870 of the electrosurgical electrode 800. One intention of the apertures 850 is to allow the insulation material on the first major face 823 to join with insulation material on the second major face 827 so as to create a seamless ring of insulation material that will tend not to lift or to delaminate. Alternative geometrical aperture configurations may also be used, such as triangular, oval, trapezoidal, or semi-circular aperture configurations.

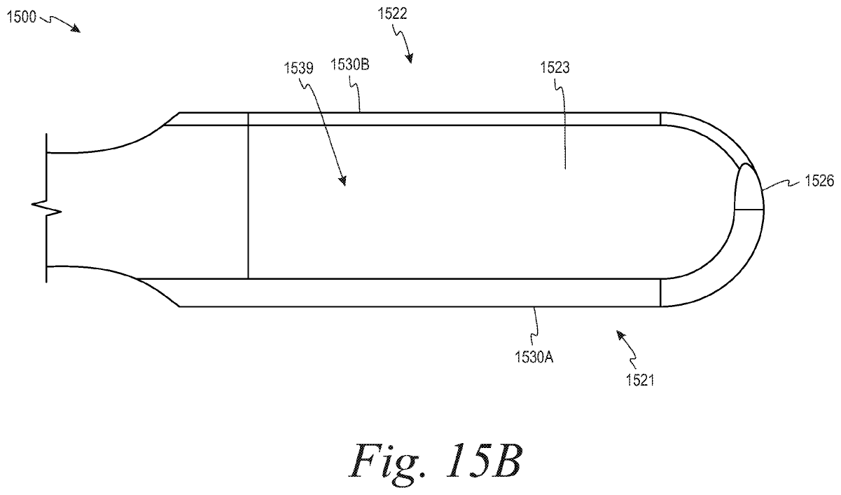

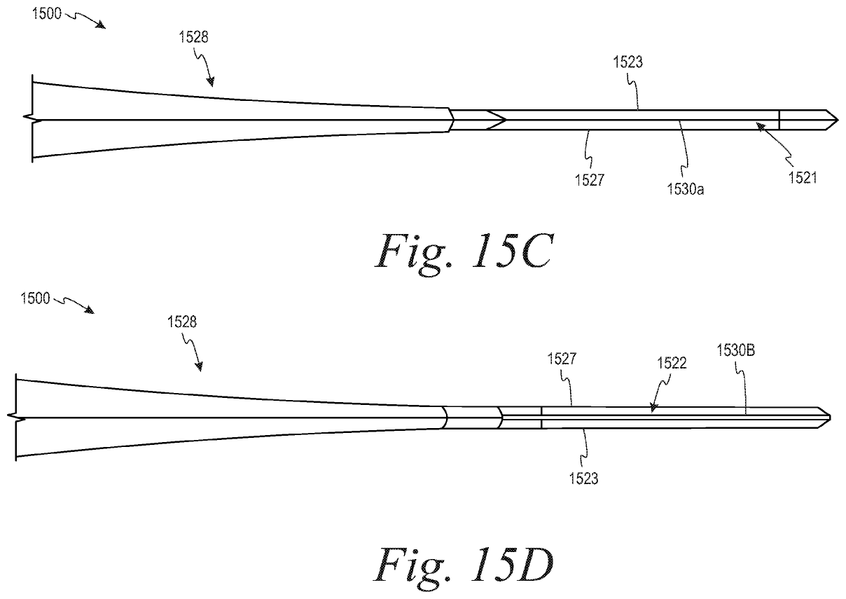

FIG. 12 illustrates a perspective view of an elongated electrosurgical electrode 900, according to an example implementation. FIG. 13 illustrates another perspective view of the elongated electrosurgical electrode 900 illustrated in FIG. 12.

Similar to the electrosurgical electrodes 400, 500, 600, 700, 800 described above, the electrosurgical electrode 800 extends in an axial direction along a longitudinal axis from a proximal electrode end 910 to a distal electrode end 920. The electrosurgical electrode 900 also includes a first lateral surface 921 and a second lateral surface 922 extending from the proximal electrode end 910 to the distal electrode end 920. The electrosurgical electrode 900 further includes a first major face 923 and a second major face 927 that each (i) extend between the proximal electrode end 910 and the distal electrode end 920, and (ii) extend between the first lateral surface 921 and the second lateral surface 922. In this arrangement, the electrosurgical electrode 900 has a length, a width, and a thickness are defined as described above.

As shown in FIGS. 12 and 13, the electrosurgical electrode 900 has a working end portion 925 in the shape of a circular head. The first major face 923 of the electrosurgical electrode 900 (FIG. 12) comprises a smooth or generally linear surface. The second major face 927 of the electrosurgical electrode 900 (FIG. 13) also comprises a smooth or general linear surface. In an arrangement, the first major face 923 and the second major face 927 are configured parallel to one another and are tapered toward one another and meet so as to define a sharp or a machined beveled outer electrode perimeter 933. This outer electrode perimeter 933 may define an edge 930 that extends along the perimeter of the electrosurgical electrode 900. In one arrangement, this edge 930 comprises a cutting edge that will not be sharp enough to mechanically cut tissue but will comprise a fine edge that will concentrate the electricity. As just one example, the fine edge may have an edge thickness in the range of approximately 70 microns to approximately 200 microns. Preferably, this fine edge may be configured for cutting and for coagulation of tissue by way of electrical energy that is received by the conductive electrode 900 as explained herein with respect to the electrosurgical systems illustrated in FIGS. 1 and 14.

In this example, the electrosurgical electrode 900 further defines a plurality of apertures 950 located generally in a central portion of the circular head and that pass through a thickness of the electrosurgical electrode 900. As just one example, the thickness of the electrosurgical electrode 900 may range from approximately 0.45 mm and approximately 0.25 mm. However, alternative thicknesses may also be used. In this illustrated arrangement, the plurality of apertures 950 are configured in an ordered series or ordered arrangement (i.e., an array of apertures) within the circular head of the working end portion 925. However, alternate aperture arrangements could also be used, such as a plurality of apertures configured in a non-ordered series or non-ordered arrangement.

In the example electrosurgical electrode 900, each of the plurality of apertures 950 comprises a circular aperture and each circular aperture comprises a generally uniform or constant circumference or radius. However, in alternative circular aperture arrangements, one or more of the circular apertures may comprise a non-uniform circumference or radius.

In this example, the electrosurgical electrode 900 further defines a first aperture 950a and a second aperture 950b. The first aperture 950a comprises a semi-circular slot that passes through a thickness of the electrosurgical electrode 900. As just one example, the thickness of the electrosurgical electrode 900 may range from approximately 0.45 mm and approximately 0.25 mm. However, alternative thicknesses may also be used. In this illustrated arrangement, the first aperture 950a propagates along a length defined by a first portion 940a of the circular head. In the example electrosurgical electrode 900, the first aperture 950a has a generally constant thickness for receiving an insulation material as described herein. However, in alternative arrangements, the first aperture 950a may comprise a non-constant thickness.

Similarly, in this illustrated example, the second aperture 950b comprises a semi-circular slot that passes through the thickness of the circular head. In this illustrated arrangement, the second aperture 950b propagates along a length defined by a second portion 940b of the circular head. In the example electrosurgical electrode 900, the second aperture 950b has a generally constant thickness for receiving an insulation material as described herein. However, in alternative arrangements, the second aperture 950b may comprise a non-constant thickness.

The first aperture 950a, the second aperture 950b, and the plurality of apertures 950 are configured to receive an insulation material, such as the insulation material illustrated and described herein with respect to FIGS. 3-5. For example, the insulation material may be coupled to or wrapped along an outer surface 970 of the electrosurgical electrode 900 so that only the first portion 940a of the edge 930, the second portion 940b of the edge 930, and a third portion 940c of the circular head remains uncovered by the insulation material.

One or more apertures 950 provided by the electrosurgical elongated electrosurgical electrode 900 will be used to help secure the insulation material along the outer surface 970 of the elongated electrosurgical electrode 900. One intention of the apertures 950 is to allow the insulation material on the first major face 923 to join with insulation material on the second major face 927 so as to create a seamless ring of insulation material that will tend not to lift or to delaminate. Alternative geometrical aperture configurations may also be used, such as triangular, oval, trapezoidal, or semi-circular aperture configurations.

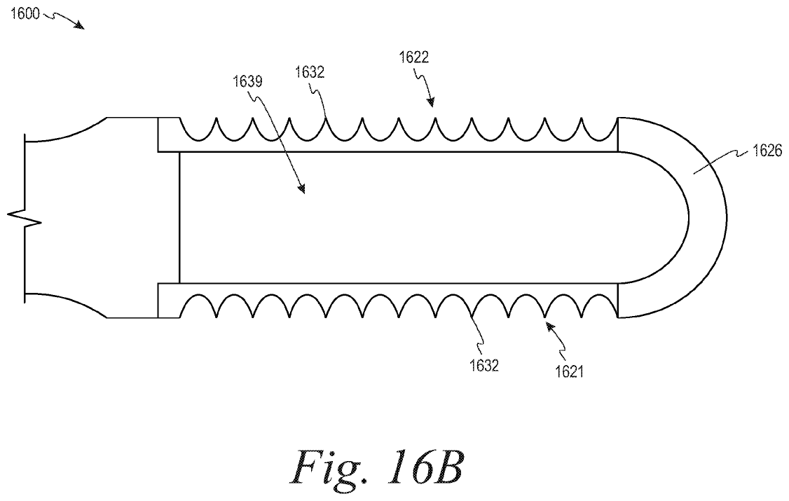

FIGS. 15A-15D illustrate an electrosurgical electrode 1500 that can be used with an electrosurgical tool (e.g., the electrosurgical tool 300 illustrated in FIG. 2), according to another example implementation. FIG. 15A illustrates a perspective view of the electrosurgical electrode 1500, FIG. 15B illustrates a plan view of the electrosurgical electrode 1500, FIG. 15C illustrates a first side view of the electrosurgical electrode 1500, and FIG. 15D illustrates a second side view of the electrosurgical electrode 1500.

Similar to the electrosurgical electrodes 400, 500, 600, 700, 800 described above, the electrosurgical electrode 1500 extends in an axial direction along a longitudinal axis from a proximal electrode end 1510 to a distal electrode end 1520. The electrosurgical electrode 1500 also includes a first lateral surface 1521 and a second lateral surface 1522 extending from the proximal electrode end 1510 to the distal electrode end 1520. The electrosurgical electrode 1500 further includes a first major face 1523 and a second major face 1527 that each (i) extend between the proximal electrode end 1510 and the distal electrode end 1520, and (ii) extend between the first lateral surface 1521 and the second lateral surface 1522. In this arrangement, the electrosurgical electrode 1500 has a length, a width, and a thickness are defined as described above.

The proximal electrode end 1510 can receive electrical energy from the electrosurgical tool. For example, the electrosurgical electrode 1500 can include a conductive material that is exposed at the proximal electrode end 1510. This can facilitate the proximal electrode end 1510 electrically coupling with the electrosurgical instrument to conduct the electrical energy from the electrosurgical instrument to the distal electrode end 1520.

The electrosurgical electrode 1500 includes a working end 1525, which is configured for cutting and coagulating tissue using the electrical energy that is received by the electrosurgical tool. As shown in FIGS. 15A-15D, the electrosurgical electrode 1500 includes a cutting edge 1530A on a first lateral surface 1521 of the electrosurgical electrode 1500 and a coagulating edge 1530B on a second lateral surface 1522 of the electrosurgical electrode 1500, which is opposite the first lateral surface 1521. The cutting edge 1530A is sharper than the coagulating edge 1530B such that a density of electrical energy is greater at the cutting edge 1530A than a density of the electrical energy at the coagulating edge 1530B when the electrical energy is applied to the electrosurgical electrode 1500. This can provide for the cutting edge 1530A achieving relatively better performance than the coagulating edge 1530B when the electrosurgical electrode 1500 is used during a cutting operation, and the coagulating edge 1530B achieving relatively better performance than the cutting edge 1530A when the electrosurgical electrode 1500 is used during a coagulating operation.

As shown in FIG. 15B, the electrosurgical electrode 1500 can additionally include a body portion 1539 extending between the first lateral surface 1521 and the second lateral surface 1522. As shown in FIGS. 15C-15D, the body portion 1539 can define the first major face 1523 and the second major face 1527, which are a pair of substantially planar surfaces between the first lateral surface 1521 and the second lateral surface 1522. In other implementations, the body portion 1539 can have a different shape. In this arrangement, the electrosurgical electrode 1500 can be in the form of an electrosurgical blade.

Within examples, the electrosurgical electrode 1500 can include at least one layer of a non-stick material covering an outer surface of the electrosurgical electrode 1500. For instance, the non-stick material can cover at least one of the body portion 1539, the cutting edge 1530A, or the coagulating edge 1530B. Accordingly, in one implementation, the non-stick material can cover the body portion 1539 but not cover the cutting edge 1530A and the coagulating edge 1530B. In another implementation, the non-stick material can cover the body portion 1539 and the cutting edge 1530A, but not cover the coagulating edge 1530B. In another implementation, the non-stick material can cover the body portion 1539 and the coagulating edge 1530B, but not the cutting edge 1530A. In another implementation, the non-stick material can cover the body portion 1539, the cutting edge 1530A, and the coagulating edge 1530B.

As examples, the layer of non-stick material can be formed from similar materials as the insulation material described above, but with lesser thickness such that the electrical energy can be applied to the tissue via the portion(s) of the electrosurgical electrode 1500 that are covered by the non-stick coating. For instance, the layer of non-stick material can include a polymeric material having a thickness that is less than 100 microns. In one example, the polymeric material can include a fluorocarbon material. For instance, the fluorocarbon material can include polytetrafluoroethylene (PTFE). Additionally or alternatively, the layer of non-stick material can include silicone, poly olefin, and/or polyamide having a thickness to permits application of electrical energy to the tissue.

The electrosurgical electrode 1500 can include one or more apertures for coupling the layer(s) of non-stick material to the electrosurgical electrode 1500, or the electrosurgical electrode 1500 can omit the apertures. As additional or alternative examples, the layer of non-stick material can be a coating (e.g., a non-stick enamel).

As shown in FIG. 15B, the electrosurgical electrode 1500 can include a distal-most end 1526. The distal-most end 1526 can provide a transition section that tapers from the relatively sharp surface of the cutting edge 1530A to the relatively blunt surface of the coagulating edge 1530B. For instance, the distal-most end 1526 can provide an edge that tapers inwardly from the coagulating edge 1530B toward the cutting edge 1530A.

As shown in FIGS. 15A, 15C, and 15D, the electrosurgical electrode 1500 can additionally include a neck portion 1528 between a proximal electrode portion and a distal electrode portion. The proximal electrode portion can have a cross-sectional size that is greater than a cross-sectional size of the distal electrode portion. This can help to allow the electrosurgical electrode 1500 to preferentially bend at the neck portion 1528 when a force is applied to the distal electrode portion. To transition from the relatively large size of the proximal electrode portion to the relatively smaller size of the distal electrode portion, the neck portion 1528 can taper inwardly toward a center axis of the electrosurgical electrode 1500 along a direction from the proximal electrode portion toward the distal electrode portion.

Although not shown in FIGS. 15A-15C, the electrosurgical electrode 1500 can additionally or alternatively include one or more apertures and/or one or more layers of insulation material as described above. The apertures(s) and/or layer(s) of insulation material can be in any of the configurations and arrangements described and illustrated above with respect to FIGS. 5-13.

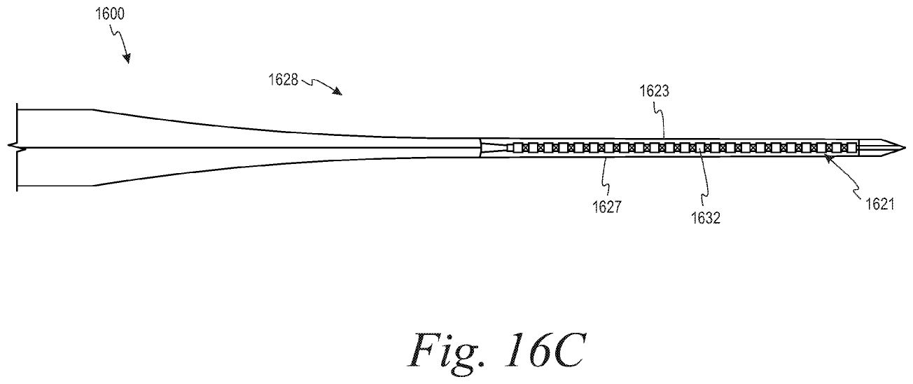

FIGS. 16A-16C illustrate an electrosurgical electrode 1600 that can be used with an electrosurgical tool (e.g., the electrosurgical tool 300 illustrated in FIG. 2), according to another example implementation. FIG. 16A illustrates a perspective view of the electrosurgical electrode 1600, FIG. 16B illustrates a plan view of the electrosurgical electrode 1600, FIG. 16C illustrates a side view of the electrosurgical electrode 1600.

Similar to the electrosurgical electrodes 400, 500, 600, 700, 800, 1500 described above, the electrosurgical electrode 1600 extends in an axial direction along a longitudinal axis from a proximal electrode end 1610 to a distal electrode end 1620. The electrosurgical electrode 1600 also includes a first lateral surface 1621 and a second lateral surface 1622 extending from the proximal electrode end 1610 to the distal electrode end 1620. The electrosurgical electrode 1600 further includes a first major face 1623 and a second major face 1627 that each (i) extend between the proximal electrode end 1510 and the distal electrode end 1620, and (ii) extend between the first lateral surface 1621 and the second lateral surface 1622. In this arrangement, the electrosurgical electrode 1600 has a length, a width, and a thickness are defined as described above.

The proximal electrode end 1610 can receive electrical energy from the electrosurgical tool. For example, the electrosurgical electrode 1600 can include a conductive material that is exposed at the proximal electrode end 1610. This can facilitate the proximal electrode end 1610 electrically coupling with the electrosurgical instrument to conduct the electrical energy from the electrosurgical instrument to the distal electrode end 1620.

The electrosurgical electrode 1600 includes a working end 1625, which is configured for cutting tissue using the electrical energy that is received by the electrosurgical tool. Within examples, the electrosurgical electrode 1600 includes at least one cutting edge 1630 on a first lateral surface 1621 and/or a second lateral surface 1622 of the electrosurgical electrode 1600. In FIGS. 16A-16C, the first lateral surface 1621 and the second lateral surface 1622 each include the cutting edge 1630. However, in other examples, the cutting edge 1630 can be provided on only one of the first lateral surface 1621 or the second lateral surface 1622.

As shown in FIGS. 16A-16C, each cutting edge 1630 includes a plurality of teeth 1632. As shown in FIG. 16B, each tooth 1632 can have a substantially triangular shape such that a base of the tooth 1632 is relatively nearer to a central axis of the electrosurgical electrode 1600 and an apex of the tooth 1632 is relatively farther from the central axis than the base. In this arrangement, the teeth 1632 can each taper to a relatively small tip point. As such, the teeth 1632 can provide for reducing a surface area of the electrosurgical electrode 1600 at the cutting edges 1630, which can help to concentrate a density of the electrical energy applied by the cutting edges 1630 to tissue during a cutting operation. This can help to improve cutting performance by, for example, reducing charring while cutting tissue.

As shown in FIG. 16B, the electrosurgical electrode 1600 can additionally include a body portion 1639 extending between the first lateral surface 1621 and the second lateral surface 1622. As shown in FIG. 16C, the body portion 1639 can define the first major face 1623 and the second major face 1727, which are in the form of a pair of substantially planar surfaces between the first lateral surface 1621 and the second lateral surface 1622. In other implementations, the body portion 1639 can have a different shape. In this arrangement, the electrosurgical electrode 1600 can be in the form of an electrosurgical blade.

In some examples, the electrosurgical electrode 1600 can include at least one layer of a non-stick material covering an outer surface of the electrosurgical electrode 1600. For instance, the non-stick material can cover at least one of the body portion 1639, the first lateral surface 1621, or the second lateral surface 1622. Accordingly, in one implementation, the non-stick material can cover the body portion 1639 but not cover the cutting edges 1630 at the first lateral surface 1621 and the second lateral surface 1622. In another implementation, the non-stick material can cover the body portion 1639 and the cutting edge 1630 at the first lateral surface 1621, but not cover the second lateral surface 1622. In another implementation, the non-stick material can cover the body portion 1639 and the cutting edge 1630 at the second lateral surface 1622, but not the first lateral surface 1621. In another implementation, the non-stick material can cover the body portion 1639 and the cutting edges 1630 at the first lateral surface 1621 and the second lateral surface 1622.

As described above, the layer of non-stick material can include a polymeric material. In one example, the polymeric material can include a fluorocarbon material. For instance, the fluorocarbon material can include polytetrafluoroethylene (PTFE). The electrosurgical electrode 1600 can include one or more apertures for coupling the layer(s) of non-stick material to the electrosurgical electrode 1600, or the electrosurgical electrode 1600 can omit the apertures. As additional or alternative examples, the layer of non-stick material can be a coating (e.g., a non-stick enamel). In other examples, the electrosurgical electrode 1600 can omit the layer of non-stick material.

As shown in FIG. 16B, the electrosurgical electrode 1600 can include a distal-most end 1626. In an example, the distal-most end 1626 can omit the plurality of teeth 1632. In another example, the distal-most end 1626 can include the plurality of teeth 1632. In one implementation in which the distal-most end 1626 include the teeth 1632, the teeth 1632 can continue to extend around the distal-most end 1626 in the same manner shown for the teeth 1632 along the first lateral surface 1621 and the second lateral surface 1622 (e.g., a size, shape, and/or spacing between the teeth 1632 on the distal-most end 1626 can be consistent with the size, shape, and/or spacing of the teeth 1632 on the first lateral surface 1621 and the second lateral surface 1622).

As shown in FIGS. 16A and 16C, the electrosurgical electrode 1600 can additionally include a neck portion 1628 between a proximal electrode portion and a distal electrode portion. The proximal electrode portion can have a cross-sectional size that is greater than a cross-sectional size of the distal electrode portion. This can help to allow the electrosurgical electrode 1600 to preferentially bend at the neck portion 1628 when a force is applied to the distal electrode portion. To transition from the relatively large size of the proximal electrode portion to the relatively smaller size of the distal electrode portion, the neck portion 1628 can taper inwardly toward a center axis of the electrosurgical electrode 1600 along a direction from the proximal electrode portion toward the distal electrode portion.

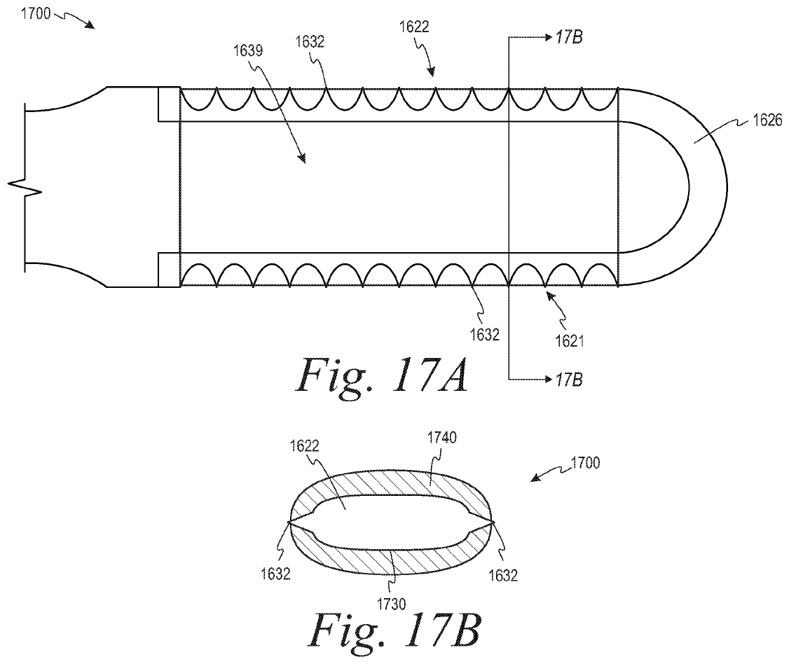

FIGS. 17A-17B illustrate an electrosurgical electrode 1700 that can be used with an electrosurgical tool (e.g., the electrosurgical tool 300 illustrated in FIG. 2), according to another example implementation. The electrosurgical electrode 1700 is substantially similar or identical to the electrosurgical electrode 1600 described above with respect to FIGS. 16A-16C, except the electrosurgical electrode 1700 includes at least one layer of insulation material 1740 on a portion of an outer surface 1730 of the electrosurgical electrode 1700. More specifically, the at least one layer of insulation material 1740 covers the body portion 1639 while the teeth 1632 on the first lateral surface 1621 and the second lateral surface 1622 protrude through the at least one layer of insulation material 1740 such that the teeth 1632 are exposed.

In one implementation, the insulation material 1740 can be a polymer heat shrink. In this implementation, the insulation material 1740 can initially be tubular. The body portion 1639 of the electrosurgical electrode 1700 can be positioned within a bore of the insulation material 1740, and then heat can be applied to shrink the insulation material 1740 onto the body portion 1639 of the electrosurgical electrode 1700. While applying the heat, the teeth 1632 can puncture the insulation material 1740 and protrude from the insulation material 1740. As such, the teeth 1632 can be exposed while a remainder of the body portion 1639 (e.g., including gaps between the teeth 1632) is covered by the insulation material 1740. In this arrangement, the insulation material 1740 can further help to concentrate a density of the electrical energy applied by the cutting edges 1630 to tissue during a cutting operation. This can help to improve cutting performance by, for example, reducing charring while cutting tissue.

As described above, the distal-most end 1626 can additionally or alternatively include the teeth 1632 in some examples. In some implementations of such examples, the at least one layer of insulation material 1740 can cover the distal-most end 1626 while exposing the teeth 1632 at the distal-most end 1626 in a similar manner to that described above.

In FIGS. 16A-17B, the teeth 1632 are generally equally spaced relative to each other. However, in another example, the teeth 1632 can have different distances between adjacent ones of the teeth 1632. For instance, a distance between a first pair of adjacent teeth 1632 can be different than a distance between a second pair of adjacent teeth 1632.

Although not shown in FIGS. 16A-17B, the electrosurgical electrode 1600, 1700 can additionally or alternatively include one or more apertures and/or one or more layers of insulation material as described above. The apertures(s) and/or layer(s) of insulation material can be in any of the configurations and arrangements described and illustrated above with respect to FIGS. 5-13.

As already noted, the disclosed electrode configurations may be used in both monopolar and bipolar applications. For example, referring now to FIG. 16, a bipolar electrosurgical system 1200 is illustrated. This bipolar electrosurgical system 1000 comprises a RF electrosurgical generator 1100 (also referred to as an electrosurgical unit or ESU). The RF electrosurgical generator 1100 utilizes a first electrosurgical electrode and a second electrosurgical electrode wire 1150 that provides for a delivery of radio-frequency (RF) current through a tissue 1300 to raise tissue temperature for cutting, coagulating, and desiccating. Such radio frequency (RF) will be current comprising rapidly alternating polarity such as on the order of approximately 0.1 to approximately 3 MHz.

The system 1000 further includes an electrosurgical tool 1400 that comprises two electrosurgical electrodes 1450a, 1450b. As explained in detail herein, example electrosurgical electrodes disclosed herein may be used with such an electrosurgical tool 1400.

Bipolar electrosurgery often requires less energy to achieve a desired tissue effect and therefor lower voltages may often be applied. Because bipolar electrosurgery has certain limited abilities to cut and coagulate large bleeding areas, bipolar electrosurgery is ideally used for those procedures where tissues can be grabbed on both sides by the electrosurgical electrodes 1450a, 1450b. Electrosurgical current in the tissue 1300 is restricted to just the tissue 1300 residing between the two electrosurgical electrodes 1450a, 1450b.

As used herein, by the term “substantially” it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide.

Different examples of the system(s), apparatus(es), and method(s) disclosed herein include a variety of components, features, and functionalities. It should be understood that the various examples of the system(s), apparatus(es), and method(s) disclosed herein may include any of the components, features, and functionalities of any of the other examples of the system(s), apparatus(es), and method(s) disclosed herein in any combination, and all of such possibilities are intended to be within the scope of the disclosure.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to explain the principles of the examples, the practical application, and to enable others of ordinary skill in the art to understand the disclosure for various examples with various modifications as are suited to the particular use contemplated.

What is claimed is:

1. An electrosurgical electrode for conveying electrical energy, the electrosurgical electrode comprising:
a proximal electrode end configured to receive electrical energy from an electrosurgical tool;
a distal electrode end;
a working end portion between the proximal electrode end and the distal electrode end, wherein the working end portion is configured for cutting or coagulation of tissue using the electrical energy that is received by the proximal electrode end;
a first lateral surface;
a second lateral surface opposite the first lateral surface;
a first major face extending between the first lateral surface and the second lateral surface on a first side of the electrosurgical electrode;
a second major face extending between the first lateral surface and the second lateral surface on a second side of the electrosurgical electrode that is opposite the first side;
one or more apertures extending entirely through a thickness of the electrosurgical electrode between the first major face and the second major face; and
at least one layer of an insulation material is coupled to an outer surface of the working end so that a first portion of the outer surface is covered by the at least one layer of the insulation material and a second portion of the outer surface is not covered by the at least one layer of the insulation material, wherein the at least one layer of the insulation material is configured to prevent applying electric current from the first portion of the outer surface to a tissue of a patient,
wherein the at least one layer of the insulation material is coupled to the outer surface at the one or more apertures.

2. The electrosurgical electrode of claim 1, wherein the at least one layer of the insulation material extends in the one or more apertures.

3. The electrosurgical electrode of any one of claims 1-2, wherein the at least one layer of the insulation material is a continuous loop extending through the one or more apertures from the first face to the second face.

4. The electrosurgical electrode of any one of claims 1-3, wherein the one or more apertures comprises a slot extending in an axial direction along a longitudinal axis between the proximal electrode end and the distal electrode end.

5. The electrosurgical electrode of any one of claims 1-4, wherein the one or more apertures comprises a plurality of apertures.

6. The electrosurgical electrode of claim 5, wherein the plurality of apertures comprises:
a first slot extending in an axial direction between the proximal electrode end and the distal electrode end; and
a second slot extending in the axial direction between the proximal electrode end and the distal electrode end,
wherein the at least one layer of the insulation material is extends (i) over the first face between the first slot and the second slot, (ii) through the second slot between the first face to the second face, (iii) over the second face between the second slot and the first slot, and (iv) through the first slot between the second face and the first face.

7. The electrosurgical electrode of claim 6, wherein the second portion of the outer surface is not covered by the at least one layer of the insulation material is (i) between the first slot and the first lateral surface and (ii) between the second slot and the second lateral surface.

8. The electrosurgical electrode of claim 5, wherein the plurality of apertures comprises an array of circular apertures.

9. The electrosurgical electrode of any one of claims 1-8, wherein the at least one layer of the insulation material comprises a polymeric material.

10. The electrosurgical electrode of claim 9, wherein the polymeric material comprises polytetrafluoroethylene (PTFE).

11. The electrosurgical electrode of any one of claims 1-10, wherein a thickness of the at least one layer of the insulation material has a thickness that is greater than approximately 100 microns.

12. The electrosurgical electrode of any one of claims 1-11, wherein the second portion is covered by a layer of a material that is configured to provide for applying electric current from the second portion of the outer surface to a tissue of a patient.

13. The electrosurgical electrode of claim 12, wherein the layer of the material is a non-stick coating.

14. The electrosurgical electrode of any one of claims 12-13, wherein the layer of the material has a thickness that is less than a thickness of the at least one layer of the insulation material.

15. The electrosurgical electrode of any one of claims 1-14, wherein the first lateral surface comprises a cutting edge,
wherein the second lateral surface comprises a coagulating edge,
wherein the cutting edge is sharper than the coagulating edge such that a density of electrical energy is greater at the cutting edge than the coagulating edge when the electrical energy is applied to the electrosurgical electrode, and
wherein the cutting edge is opposite the coagulating edge.

16. The electrosurgical electrode of claim 15, wherein the cutting edge has a thickness of approximately 70 microns to approximately 200 microns.

17. The electrosurgical electrode of any one of claims 1-16, further comprising a plurality of teeth on at least one of the first lateral surface or the second lateral surface.

18. The electrosurgical electrode of claim 17, wherein a distal-most end of the electrosurgical electrode comprises the plurality of teeth.

19. An electrosurgical electrode for conveying electrical energy, the electrosurgical electrode comprising:
a proximal electrode end configured to receive electrical energy from an electrosurgical tool;
a distal electrode end;
a working end portion between the proximal electrode end and the distal electrode end, wherein the working end portion is configured for cutting or coagulation of tissue using the electrical energy that is received by the proximal electrode end;
a first lateral surface;
a second lateral surface opposite the first lateral surface;
a first face extending between the first lateral surface and the second lateral surface on a first side of the electrosurgical electrode;
a second face extending between the first lateral surface and the second lateral surface on a second side of the electrosurgical electrode that is opposite the first side; and
a plurality of teeth on at least one of the first lateral surface or the second lateral surface, wherein the plurality of teeth can each taper to a respective tip point.

20. The electrosurgical electrode of claim 19, further comprising at least one layer of an insulation material covering a body portion of the electrosurgical electrode,
wherein the plurality of teeth on the first lateral surface and the second lateral surface protrude through the at least one of the insulation material such that the tip points of the plurality of teeth are exposed.
